(19) United States
(12) Patent Application Publication
(10) Pub. No.: US 2013/0280324 A1
(43) Pub. Date: Oct. 24, 2013

(54) SUSTAINED RELEASE PHARMACEUTICAL COMPOSITIONS COMPRISING PREGABALIN

(71) Applicants: Rajesh Jain, New Delhi (IN); Sarabjit Singh, New Delhi (IN); Shivanand Puthli, New Delhi (IN)

(72) Inventors: Rajesh Jain, New Delhi (IN); Sarabjit Singh, New Delhi (IN); Shivanand Puthli, New Delhi (IN)

(73) Assignee: PANACEA BIOTEC LTD., New Delhi (IN)

(21) Appl. No.: 13/824,188

(22) PCT Filed: Sep. 15, 2011
(86) PCT No.: PCT/IN11/00638

§ 371 Date: Mar. 15, 2013

(30) Foreign Application Priority Data
Sep. 17, 2010 (IN) 2597/MUM/2010

(51) Int. Cl. A61K 9/00; A61K 47/26; A61K 31/197

(52) U.S. Cl. CPC A61K 9/0002; A61K 31/197; A61K 47/26
USPC 424/452; 514/561

(57) ABSTRACT

The present invention relates to stable once daily sustained release pharmaceutical compositions comprising pregabalin or pharmaceutically acceptable salts thereof and a pharmaceutically acceptable excipient wherein pharmaceutical composition is bioequivalent to conventional immediate release formulation of pregabalin administered twice daily. The present invention further relates to a composition comprising pregabalin and sugar esters as release retarding agent for maintaining uniform release rate of the drug and process for the preparation of such oral sustained release formulations.

### FIELD OF THE INVENTION

The present invention relates to a sustained release oral pharmaceutical formulation comprising Pregabalin and at least one pharmaceutically acceptable excipient. The present invention more specifically relates to a once daily sustained release pharmaceutical composition comprising pregabalin or pharmaceutically acceptable salts thereof and a pharmaceutically acceptable excipient wherein pharmaceutical composition is bioequivalent to conventional immediate release formulation of pregabalin (LYRICA®) administered twice daily. The present invention further relates to the composition comprising Pregabalin and sugar esters as release retarding agent for maintaining uniform release rate of the drug and process for the preparation of such oral sustained release formulations.

### BACKGROUND OF THE INVENTION

Pregabalin is disclosed in U.S. Pat. No. 6,197,819. Further, U.S. Pat. No. 6,197,819 also has generic disclosure of pharmaceutical compositions comprising pregabalin. U.S. Pat. No. 5,563,175 describes the use of pregabalin in the treatment of seizure disorders. U.S. Pat. No. 6,117,906 discloses the use of pregabalin in treating anxiety, while U.S. Pat. No. 6,001,876 discloses its use in treating pain. U.S. Pat. Nos. 6,663,175, 5,599,973, 5,608,090, 5,684,189, 5,710,304, 5,616,793, 5,629,447, 5,637,767, 5,840,956, 6,046,353, 6,028,214 disclose processes for preparation of pregabalin and intermediates used in these processes.

Pregabalin was approved in the United States on Dec. 30, 2004, an immediate release dosage form for use in the treatment of diabetic peripheral neuropathy, postherpetic neuralgia, and as an adjunctive treatment for partial onset seizures in adults.

Pregabalin is currently available as immediate release Lyrica® in 25, 50, 75, 100, 150, 200, 225, and 300 mg hard shell capsules and is administered in patients two or three times daily (BID or TID).

The recommended dose of pregabalin is 100 mg three times a day (300 mg/day) for the treatment of neuropathic pain associated with diabetic peripheral neuropathy and post herpetic neuralgia.

In the management of Fibromyalgia, the recommended dose of LYRICA for fibromyalgia is 300 to 450 mg/day. Begin dosing at 75 mg two times a day (150 mg/day). The dose may be increased to 150 mg two times a day (300 mg/day) within 1 week based on efficacy and tolerability. Pregabalin at doses of 150 to 600 mg/day is recommended for adjunctive therapy for adult patients with partial onset seizures.

Multiple administrations associated with conventional immediate release formulations leads to substantial fluctuations in the plasma concentration of the drug. It is therefore desirable that the administered drug should have a relatively constant plasma concentration throughout the day. The convenience of once a day dosing improves patient compliance, especially for elderly patients and for patients on multiple medications. Once per day dosing may also lessen or prevent potentially undesirable dose-related effects by reducing peak blood levels. Extended release also permits widening of the absorption window.

Once daily dosing of pregabalin, however, presents numerous challenges as pregabalin is not absorbed uniformly in the gastrointestinal (GI) tract. Pregabalin is absorbed in the small intestine and in the ascending colon in humans.

Providing once a day formulation which is bio equivalent to twice a day immediate release formulation in terms of peak plasma exposure and total exposure poses several challenges. Various approaches have been tried out for developing a once daily dosage form of pregabalin.

WO2007/052125A2 relates to a pharmaceutical composition comprising pregabalin, and matrix forming agent and a swelling agent, the matrix-forming agent comprising polyvinyl acetate and polyvinylpyrrolidone, and the swelling agent comprising cross-linked polyvinylpyrrolidone, wherein the pharmaceutical composition is adapted for once-daily oral dosing. It further discloses once a day formulation of pregabalin which has lower Cmax (peak exposure) and comparable total exposure (AUC) when compared with IR formulations of pregabalin.

US2005/0163848A1 relates to a complex comprised of pregabalin and a transport moiety, such as an alkyl sulfate. The complex has an enhanced absorption in the gastrointestinal tract, particularly the lower gastrointestinal tract. The compositions and dosage forms prepared using the complex, provide for absorption by the body of the drug through a period of ten to twenty-four hours, thus enabling a once-daily dosage form for pregabalin.

US2002/0119197A1 relates to pharmaceutical dosage form comprising a central core including a pharmaceutical agent in a controlled-release composition, said core having two exposed opposite end surfaces and a peripheral surface at an outer edge of said core extending between said two opposed end surfaces, said peripheral edge surrounded by a diffusion-limiting sleeve, wherein said sleeve limits the diffusion of fluids into said core. Core comprises pharmaceutical agent (pregabalin) embedded in a matrix (hydroxypropyl methylcellulose, polyvinyl pyrrolidone etc.) Diffusion-limiting sleeve material comprises at least one of ethylcellulose and polymethacrylate.

WO2009/066325A1 relates to controlled release pharmaceutical compositions comprising a water soluble or water swellable inert core, a layer of therapeutically effective amount of pregabalin and a release controlling layer comprising hydrophobic release controlling agents. Extended release coatings comprising hydroxy propyl methyl cellulose (HPMC) and ethylcellulose have been exemplified.

It has been found to be difficult to formulate sustained release formulations comprising highly soluble medicaments such as pregabalin and hydrophilic polymeric agents like hydroxyalkylcelluloses, alkylcelluloses, carboxyalkylcelluloses and polyvinylpyrrolidone.

Firstly active ingredients which are soluble in water when formulated with polymeric matrices like hydroxyalkyl celluloses, polyvinyl pyrrolidone tend to generate a sustained release product which is susceptible to a phenomenon known as dose dumping. In dose dumping the release rate is premature and exaggerated i.e. release of the active ingredient is delayed for a time but once release begins to occur the rate of release is very high.

Moreover, fluctuations tend to occur in the plasma concentrations of the active ingredient which increases the likelihood of toxicity. It was also found to be difficult to achieve the desired dissolution profiles or to control the rate of release of the soluble medicament.

None of the above mentioned prior arts addressed the need of a once a day sustained release dosage form that provides stable plasma levels while achieving bioequivalence to conventional immediate release formulations of pregabalin with respect to peak exposure and total exposure.

Accordingly, a need exists for stable sustained release compositions comprising pregabalin or a pharmaceutically acceptable salt, which overcome, or at least alleviate, one or more of the above described difficulties and which further provides the advantageous property of allowing the active medicament to be administered once a day, while ensuring stable plasma levels, uniform drug release and bioequivalence with conventional immediate release formulations administered twice a day.

### OBJECTS OF THE INVENTION

It is an object of the present invention to provide stable once a day sustained release compositions of pregabalin having extended therapeutic plasma level over a period of 24 hours while ensuring bioequivalence with conventional immediate release formulations administered twice a day.

The present invention further relates to a sustained release pharmaceutical composition comprising pregabalin or pharmaceutically acceptable salts thereof, a release controlling agent and pharmaceutically acceptable excipient(s) wherein pharmaceutical composition comprising 300 mg pregabalin administered once daily is bioequivalent to conventional immediate release composition of pregabalin of 150 mg (Lyrica® 150 mg) administered twice daily

In another aspect the present invention relates to sustained release pharmaceutical compositions comprising pregabalin or pharmaceutically acceptable salts thereof, a release controlling agent and pharmaceutically acceptable excipient(s) wherein pharmaceutical composition comprising pregabalin administered once daily is bioequivalent when compared in terms of total exposure (AUC) to conventional immediate release composition of pregabalin administered thrice daily.

Yet another object of the invention is to provide stable sustained release pharmaceutical compositions comprising therapeutically effective amount of pregabalin including its base or pharmaceutically acceptable complexes, salts, polymorphs, hydrates, solvates, enantiomers or racemates as active ingredient, a release controlling agent(s) and optionally other pharmaceutically acceptable excipients thereof.

Yet another object of the present invention is to provide stable sustained release pharmaceutical compositions comprising therapeutically effective amount of pregabalin including its base or pharmaceutically acceptable complexes, salts, polymorphs,hydrates,solvates,enantiomers or racemates as active ingredient and sucrose fatty acid ester(s) as a release controlling agent(s) and optionally other pharmaceutically acceptable excipients thereof.

Yet another object of the present invention is to provide sustained release pharmaceutical compositions comprising pregabalin or pharmaceutically acceptable complexes, salts, polymorphs, hydrate solvates, enantiomers or racemates thereof wherein the composition comprises pregabalin embedded in a sucrose fatty acid ester matrix together with one or more pharmaceutically acceptable excipients.

Yet another object of the present invention relates to the method of treating neuropathy, post herpetic neuralgia, adjunctive therapy for adult patients with partial onset seizures and fibromyalgia in mammals comprising administering a sustained release pharmaceutical composition comprising Pregabalin or pharmaceutically acceptable salts thereof, wherein the effect is sustained for an extended period of time.

Yet another object of the present invention relates to the process of preparation of sustained release pharmaceutical compositions wherein the process comprises mixing pregabalin; sucrose fatty acid ester and optionally other pharmaceutical excipients, granulating the resultant blend compressing the granules into a tablet or filling the granules into a hard gelatin capsule.

Yet another object of the present invention relates to the sustained release pharmaceutical compositions wherein the dissolution of drug is for a prolonged period of time i.e. from at least 6 to 24 hours in aqueous media.

Yet another object of the present invention is to provide sustained release pharmaceutical compositions in which the compositions exhibit an in vitro release of at least 25% of pregabalin after 6 hours in an aqueous medium.

A further object of the present invention is to provide sustained release pharmaceutical compositions, in which the compositions exhibit an in vitro release of at least 50% of pregabalin after 12 hours in an aqueous medium.

A further object of the present invention is to provide sustained release pharmaceutical compositions, in which the compositions exhibit an in vitro release of at least 75% of pregabalin after 24 hours in an aqueous medium.

Yet another object of the present invention relates to a sustained release pharmaceutical composition comprising pregabalin or pharmaceutically acceptable salts thereof and pharmaceutically acceptable excipient(s) wherein pharmaceutical composition administered once daily provides peak plasma concentrations of pregabalin ranging from 1 μg/ml to 10 μg/ml.

Yet another object of the present invention relates to a sustained release pharmaceutical composition comprising pregabalin or pharmaceutically acceptable salts thereof and pharmaceutically acceptable excipient(s) wherein pharmaceutical composition administered once daily exhibits an AUC of pregabalin ranging from 10 μg/ml to 100 μg/ml.

### BRIEF DESCRIPTION OF DRAWINGS

FIG. 1 shows a release profile of sustained release dosage forms of pregabalin of example 6, in Type II USP apparatus, 750 ml of 0. IN HCl followed by 1000 ml of pH 6.8 phosphate buffer, 50 rpm.

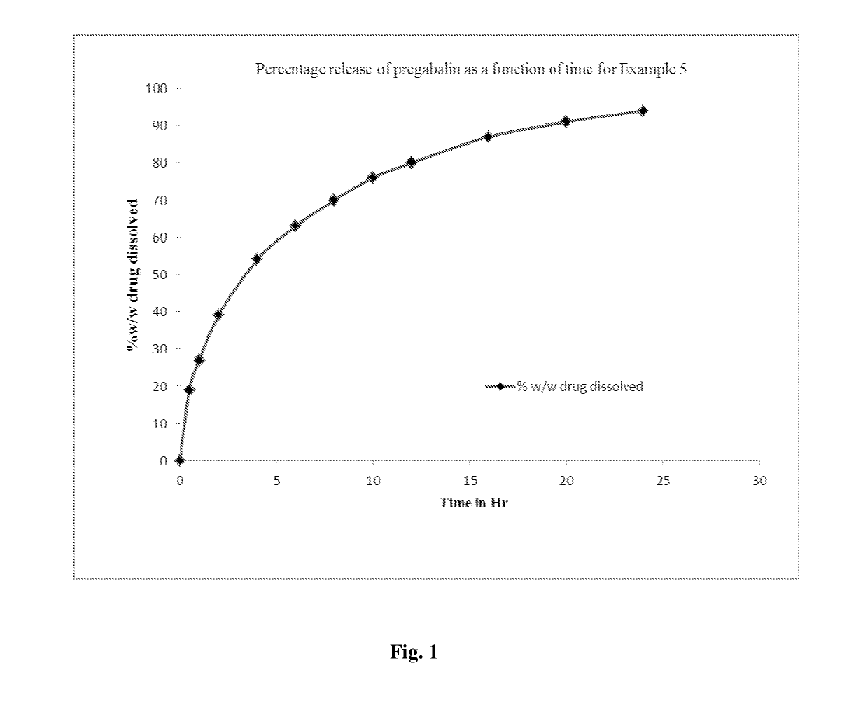

### DETAILED DESCRIPTION OF THE INVENTION

Pharmaceutical composition” refers to the combination of one or more drug substances and one or more excipients”

“Drug product,” “pharmaceutical dosage form,” “dosage form,” “final dosage form” and the like, refer to a pharmaceutical composition that is administered to a subject in need of treatment and generally may be in the form of tablets, capsules, sachets containing powder or granules, pellets, liquid solutions or suspensions, patches, and the like.

“About” will be understood by persons of ordinary skill in the art and will vary to some extent on the context in which the term is used. If there are uses of the term which are not clear to persons of ordinary skill in the art given the context in which it is used, “about” will mean up to plus or minus 10% of the particular term.

The term ‘pharmaceutically acceptable excipient’ according to the present invention means, but not limited to, any inactive ingredient which is required for the formulation of Pregabalin in a suitable dosage form. Particularly the excipient includes, but not limited to, diluents, carriers, fillers, bulking agents, binders, disintegrants, polymer, lubricant, glidant, surface active agents, stabilizers, absorption accelerators, flavoring agents, preservatives, antioxidants, buffering agents, and any other excipient commonly used in the pharmaceutical industry.

The term “sustained release compositions” herein refers to any composition or dosage form which comprises an active drug and which is formulated to provide a longer duration of pharmacological response after administration of the dosage form than is ordinarily experienced after administration of a corresponding immediate release composition comprising the same drug in the same amount. Sustained release compositions include, inter alia, those compositions described elsewhere as “extended release”, “delayed release”, “controlled release”, “prolonged release”, “programmed release”, “modified release”, “time release” and/or “rate controlled” compositions or dosage forms.

Release controlling agent refers to those substances that retard or delay the release of drug from the matrix and provide a longer duration of therapeutic response after administration of the dosage form than is typically experienced after administration of a corresponding immediate release composition. Release controlling agents as described herein include cellulose ethers, cellulose esters, acrylates, waxes, gums, glyceryl fatty acid esters and sucrose fatty acid esters.

Cmax or peak plasma exposure as described herein refers to the point of maximum concentration of drug in plasma and is expressed in μg/ml

Area under curve (AUC) or total plasma exposure refers to the total integrated area under plasma level time profile and expresses the total amount of drug that comes into systemic circulation after administration and is expressed in μg/ml

The term “bioequivalent” means the absence of a significant difference in the rate and extent to which the active ingredient or active moiety in pharmaceutical equivalents or pharmaceutical alternatives becomes available at the site of drug action when administered at the same molar dose under similar conditions in an appropriately designed study. Two compositions can be considered as “bioequivalent” if the 90% Confidence Interval of the relative mean Cₘₐₓ and AUC of the test to reference is within 80.00% to 125.00%.

### ABBREVIATIONS

HPMC: Hydroxy propyl methyl cellulose

CI: Confidence Interval

Cmax: Peak plasma exposure

AUC: Total plasma exposure

HLB: Hydrophilic Lipophilic Balance

HPLC: High-pressure liquid chromatography

USP: United States Pharmacopoeia

RH: Relative humidity

CV: Coefficient of variation

SD: Standard Deviation

RPM: Revolutions per minute

Pregabalin, or (S)-3-(aminomethyl)-5-methylhexanoic acid, binds to the calcium channel alpha-2-delta (a25) subunit and is related to endogenous inhibitory neurotransmitter gamma-amino butyric acid (GABA), which is involved in brain neuronal activity. Pregabalin is indicated for the management of neuropathic pain associated with diabetic peripheral neuropathy or post herpetic neuralgia; as an adjunctive therapy for adult patients with partial onset seizures and for the treatment of generalized anxiety disorder in adults. The dosage forms of the invention optionally may comprise pharmaceutically acceptable complexes, salts, polymorphs, hydrates, solvates, enantiomers or racemates of pregabalin.

It has been surprisingly found by the inventors that present invention provides stable once a day sustained release compositions of pregabalin which are bioequivalent with conventional immediate release formulations administered twice a day and advantageously provides significantly less plasma variation of drug as compared to other hydrophilic systems.

An embodiment of the present invention provides stable sustained release compositions comprising therapeutically effective amount of pregabalin including its base or pharmaceutically acceptable complexes, salts, polymorphs, hydrates, solvates, enantiomers or racemates as active ingredient, a release controlling agent(s) and optionally other pharmaceutically acceptable excipients thereof.

In another embodiment of the present invention pregabalin or pharmaceutically acceptable complexes, salts, polymorphs, hydrates, solvates, enantiomers or racemates thereof is present in an amount from 20% to 90% by weight of dosage form, more preferably 20% to 75% by weight of solid dosage form.

In yet another embodiment of the present invention relates to a sustained release compositions of pregabalin comprising particle of pregabalin having a size less than 100 microns.

In another embodiment the release controlling agent is selected from the group consisting of cellulose ethers, cellulose esters, acrylic acid polymers, waxes, gums, glyceryl fatty acid esters and sucrose fatty acid esters.

Non-limiting examples of cellulose ethers useful according to the present invention are alkyl celluloses specifically ethylcellulose, methylcellulose, ethylmethylcellulose, hydroxyalkylcelluloses specifically; hydroxy propyl cellulose, hydroxypropyl methyl cellulose, hydroxy ethylmethyl celluloses and carboxyalkylcelluloses. Non limiting examples of cellulose esters useful according to the present invention are cellulose acetate, cellulose triacetate, cellulose acetate propionate, cellulose acetate butyrate, cellulose acetate pthalate. Acrylic acid polymers are selected from the group comprising but not limited to poly(methyl methacrylate), poly(ethyl methacrylate), poly(butyl methacrylate), poly(isobutyl methacrylate). Non limiting examples of glyceryl fatty acid esters are glyceryl monostearate, glyceryl behenate, and glyceryl monooleate. Sucrose fatty acid esters selected from the group comprising but not limited to, sucrose stearate, sucrose distearate, sucrose palmitate, sucrose oleate, sucrose laurate, sucrose behenate, sucrose erucate. Non-limiting examples of waxes used according to the present invention include beeswax, carnauba wax, paraffin wax; microcrystalline wax. Non limiting examples of gums useful according to the present invention are alginates and carrageenan.

In preferred embodiment sucrose fatty acid esters are used as release controlling agent. Sucrose esters are esters of sucrose and fatty acids derived from edible fats and oils and are tasteless, odourless and non-toxic materials. As sucrose has eight free hydroxyl groups it can be esterified with upto eight fatty acids to form esters ranging from monoesters to octaesters. Differences in fatty acid type and degree of esterification provide sucrose esters with hydrophilic lipophilic balance values ranging from 0 to 16. As they comprise both hydrophilic groups from hydroxyl heads of sucrose and lipophilic parts from fatty acid tails, they have a unique property that tolerates temperature variation.

It has also been surprisingly found by the inventors of present invention that a sustained release carrier comprising sugar esters is sufficient to provide suitable sustained release of a highly soluble medicament to provide therapeutically effective blood levels for extended periods of time i.e. from 6 to about 24 hours. A sustained release effect prevailed in dosage forms containing sucrose esters with HLB values of 3-16. Sucrose fatty acid esters exhibited atypical release kinetics showing contribution of both swelling and erosion systems and this contributed to uniform drug release over a prolonged period of time.

In another embodiment the amount of sugar ester used is 20% to 90% by weight of the solid dosage form and more preferably 30%-60% by weight of solid dosage form. The sucrose ester according to the present invention may be selected from the group comprising but not limited to sucrose stearate, sucrose palmitate, sucrose oleate, sucrose laurate, sucrose behenate, and sucrose erucate. Sucrose esters are commercially available under trademark SURFHOPE® SE PHARMA from Mitsubishi Kagaku, Tokyo and in many grades depending upon the fatty acid and HLB value such as sucrose stearate (S-270, S-970, S-1570 and S-1670), sucrose laurate (L-1695), sucrose palmitate (P-1570 and P-1670).

In a specific embodiment sucrose stearate of grade S1570 is used in an amount of 40% to 55% by weight of solid dosage form.

In another specific embodiment sucrose laurate of grade D1216 is used in an amount from 60% to 75% by weight of solid dosage form.

Diluents increase the bulk of a solid pharmaceutical composition, and may make a pharmaceutical dosage form containing the composition easier for the patient and care giver to handle. Non-limiting examples of diluents useful according to the present invention include calcium carbonate, calcium phosphate (dibasic or tribasic), calcium sulfate, dextrates, dextrin, dextrose excipient, fructose, kaolin, lactitol, anhydrous lactose, lactose monohydrate, maltose, mannitol, sorbitol, sucrose, starch, pregelatinized starch, talc and the like or combinations thereof.

In an embodiment the amount of diluent present may vary from about 0% to about 85% by weight of solid dosage form and more preferably from about 0-60% by weight of solid dosage form. In a preferred embodiment lactose monohydrate is used as a diluent in an amount from 8%-50% by weight of solid dosage form.

In yet another embodiment carriers for use according to the present invention may include, but are not limited to, hydrophilic or hydrophobic polymers, lactose, white sugar, sodium chloride, glucose, urea, starch, calcium carbonate, calcium sulphate, kaolin, crystalline cellulose, silicic acid, and the like or combinations thereof.

A binder (also sometimes called adhesive) is added to a drug-filler mixture to ensure that granules and tablets can be formed with the required mechanical strength. Binders can be added to the formulation in different ways: (i) as a dry powder, which is mixed with other ingredients before wet agglomeration, (ii) as a solution, which is used as agglomeration liquid during wet agglomeration, and is referred to as a solution binder, and (iii) as a dry powder, which is mixed with the other ingredients before compaction. In this form the binder is referred to as a dry binder. Both solution binders as well as dry binder have been used herein.

In another embodiment binders used according to the present invention include, but are not limited to, acacia, alginic acid, carbomer, sodium carboxymethylcellulose, dextrin, ethylcellulose, gelatin, glucose, guar gum, hydroxypropyl methyl cellulose, hydroxypropyl cellulose, maltose, methylcellulose, povidone, starch, gelatin, methylcellulose, polyethylene oxide and the like or combinations thereof. The amount of binder present may vary from about 0% to 80% by weight of solid dosage form and preferably from about 0% to 40% by weight of solid dosage form. In a preferred embodiment hydroxypropylmethyl cellulose is used as a binder in an amount of 1%-20% by weight of solid oral dosage form.

Lubricants are added to pharmaceutical formulations to ensure that tablet formation and ejection can occur with low friction between the solid and the die wall. High friction during tabletting can cause a series of problems, including inadequate tablet quality (capping or even fragmentation of tablets during ejection, and vertical scratches on tablet edges) and may even stop production. Non-limiting examples of lubricants useful according to the present invention are hydrogenated vegetable oils, stearic acid, polyethylene glycol (molecular weight 4000 and higher), calcium stearate, glyceryl behenate, magnesium stearate, mineral oil, polyethylene glycol, sodium stearyl fumarate, stearic acid, talc, vegetable oil, sodium lauryl sulfate, and zinc stearate or the combinations thereof.

In an embodiment lubricants may be used in an amount from about 0.25% to about 10% by weight of solid dosage form more preferably from 0.25% to 5% by weight of solid dosage form.

In a preferred embodiment magnesium stearate is used as a lubricant in an amount from 0.25% to 5% by weight of solid dosage form.

Glidants can be added to improve the flowability of a pharmaceutical composition and improve the accuracy of dosing. In an embodiment of the present invention glidants used in the composition include colloidal silicon dioxide, magnesium trisilicate, powdered cellulose, starch, talc, and tribasic calcium phosphate and the like or combinations thereof. The amount of glidant used may vary from about 0% to 5% by weight of solid dosage form and more preferably from 0% to 2% by weight of solid dosage form In a preferred embodiment talc is used as a diluent in an amount from 0% to 2% by weight of tablet.

In an embodiment solid dosage forms according to the present invention may be prepared by any conventional techniques for example dry compaction (slugging), direct compression, wet granulation; melt granulation and extrusion-spheronization.

In an embodiment solid dosage forms according to the present invention may be prepared by a process comprising the following steps:

Step 1: Pregabalin and sugar esters were mixed together and sifted

Step 2: Blend obtained in step 1 was granulated with hypromellose solution in dichloromethane and resulting granules were dried.

Step 3: Magnesium stearate was mixed with the dried granules

Step 4: The resulting granules were compressed on a rotary tablet compression machine or filed into capsules.

In another embodiment solid dosage forms according to the present invention may be prepared by a process comprising the following steps

Step 1: Mixing pregabalin; sucrose fatty acid ester and optionally other pharmaceutical excipients

Step 2: Making slugs of resultant mixture as obtained in step 1

Step 3: Breaking down the slugs obtained in step 3 to obtain the granules of desired size

Step 4: Optionally mixing the granules of step 3 with other excipients to obtain final blend.

Step 5: Compressing the resultant blend of step 4 to obtain tablets or filling such final blend into hard gelatin capsules.

In another embodiment of the present invention the sustained release properties may be demonstrated by monitoring the dissolution of active ingredient. The dissolution of active ingredient may be monitored by using standard procedures well known to those skilled in the art. Examples of dissolution test procedures include Rotating basket method (Apparatus USP type I) or Paddle method (Apparatus USP type II) as disclosed in U.S. Pharmacopoeia (USP). Such procedures include those in which the formulation is immersed in an aqueous medium like water hydrochloric acid or other buffer solution with varying pH. Aliquots of the medium are withdrawn at various time points over a period of 24 hours. The aliquots are then analyzed using HPLC with UV detection to determine the concentration of dissolved active ingredient using standard methodology. The formulation according to the present invention releases the active ingredient over prolonged period of time from at least 6 to 24 hours in aqueous media.

In an embodiment sustained release pharmaceutical compositions according to the present invention exhibit an in vitro release of at least 25% of pregabalin after 6 hours in an aqueous medium.

In yet another embodiment sustained release pharmaceutical compositions according to the present invention, exhibit an in vitro release of at least 50% of pregabalin after 12 hours in an aqueous medium.

In yet another embodiment sustained release pharmaceutical compositions according to the present invention exhibit an in vitro release of at least 75% of pregabalin after 24 hours in an aqueous medium.

The following examples are merely illustrative of the present invention and they should not be considered as limiting the scope of the invention in any way, as these examples and other equivalents thereof will become more apparent to those versed in the art in the light of the present disclosure

### COMPARATIVE EXAMPLE 1A

| | |
|---|---|
| Ingredients | Mg/dosage form |

Intra-granular

| | |
|---|---|
| Pregabalin | 300.00 |
| Lactose anhydrous | 431.00 |
| (Hydroxypropyl methylcellulose K100M) | 152.00 |
| (Povidone K29/32) | 38.00 |
| Compritol | 24.00 |

Extra-granular

| | |
|---|---|
| Talc | 5.00 |
| Total | 950.00 |

Pregabalin, Lactose anhydrous, Hypromellose, Polyvinylpyrrolidone and were mixed together and sifted. The resultant blend obtained was mixed with Compritol. Talc was mixed with the resultant blend and compressed on rotary tablet compression machine.

### EXAMPLES 1-2

| | | |
|---|---|---|
| Ingredients (mg) | Ex 1 | Ex 2 |

Intragranular

| | | |
|---|---|---|
| Pregabalin | 300 | 300 |
| Sucrose laurate | — | 300 |
| Sucrose stearate | 300 | |
| Hypromellose | 12.80 | 12.80 |
| Dichloromethane* | q.s | q.s |

Extragranular

| | | |
|---|---|---|
| Magnesium stearate | 30.64 | 30.64 |
| Total | 643.44 | 643.44 |

*Evaporates during the process of manufacturing

Pregabalin and sugar esters were mixed together and sifted. Blend obtained in step 1 was granulated with Hypromellose solution in dichloromethane and resulting granules were dried. Magnesium stearate was mixed with the dried granules and compressed on rotary tablet compression machine.

### EXAMPLES 3-7

| | | | | | |
|---|---|---|---|---|---|
| Ingredients (mg) | Ex 3 | Ex 4 | Ex 5 | Ex 6 | Ex 7 |

Intragranular

| | | | | | |
|---|---|---|---|---|---|
| Pregabalin | 300.00 | 300.00 | 300.00 | 300.00 | 300.00 |
| Sucrose laurate | — | — | — | 860.00 | — |
| Sucrose stearate | — | — | 280.00 | — | — |
| Sucrose distearate | 300.00 | 327.00 | — | — | 91.40 |
| Lactose anhydrous | — | 110.50 | 198.00 | 45.00 | 43.60 |
| Lactose monohydrate | 135.00 | — | — | — | — |

Extragranular

| | | | | | |
|---|---|---|---|---|---|
| Talc | 15.00 | 7.5 | 7.00 | 7.00 | 7.00 |
| Magnesium stearate | 6.00 | 15 | 15.00 | 15.00 | 15.00 |
| Total | 756.00 | 760.00 | 800.00 | 1227.00 | 457.00 |

Pregabalin, lactose, and sucrose fatty acid ester were mixed together and sifted. This blend was then subjected to slugging and resulting slugs were broken down and sifted through the sieve. Magnesium stearate and talc were mixed with the granules. The resulting blend was compressed on rotary tablet compression machine.

### EXAMPLES 8-10

| | | | |
|---|---|---|---|
| Ingredients (mg) | Ex 8 | Ex 9 | Ex. 10 |

Intragranular

| | | | |
|---|---|---|---|
| Pregabalin | 150.00 | 150.00 | 150.00 |
| Sucrose laurate | 140.00 | — | — |
| Sucrose stearate | — | 45.00 | — |
| Sucrose distearate | — | — | 430 |
| Lactose anhydrous | 99 | 22.5 | 22.5 |

Extragranular

| | | | |
|---|---|---|---|
| Talc | 3.50 | 3.50 | 3.50 |
| Magnesium stearate | 7.50 | 7.50 | 7.50 |
| Total | 400.00 | 228.50 | 613.50 |

Pregabalin, lactose, and sucrose fatty acid ester were mixed together and sifted. This blend was dry granulated/compacted and resulting compacts were milled. Talc was mixed with the granules followed by lubrication with magnesium stearate. The resulting blend was compressed on rotary tablet compression machine.

### EXAMPLES 11-12

| | | |
|---|---|---|
| Ingredients (mg) | Ex 11 | Ex 12 |

Intragranular

| | | |
|---|---|---|
| Pregabalin | 300.00 | 300.00 |
| Ethylcellulose | 300.00 | — |
| Lambda Carrageenan | — | 327.00 |
| Lactose anhydrous | 135.00 | 110.50 |

Extragranular

| | | |
|---|---|---|
| Talc | 15.00 | 7.50 |
| Magnesium stearate | 6.00 | 15.00 |
| Total | 756.00 | 760.00 |

Pregabalin, lactose, and sustained release agent (Ethylcellulose and carageenan as exemplified in examples 11 and 12 respectively) were mixed together and sifted. This blend was then subjected to slugging and resulting slugs were broken down and sifted through the sieve. Magnesium stearate and talc were mixed with the granules. The resulting blend was compressed on rotary tablet compression machine

### EXAMPLE 13

TABLE 1
Amount of pregabalin released from the
dosage form as a function of time.

| Time (hr) | % w/w drug dissolved |
|---|---|
| 0 | 0 |
| 0.5 | 19 |
| 1 | 27 |
| 2 | 39 |
| 4 | 54 |
| 6 | 63 |
| 8 | 70 |
| 10 | 76 |
| 12 | 80 |
| 16 | 87 |
| 20 | 91 |
| 24 | 94 |

The amount of drug released from the dosage form as exemplified in example 5 is determined by paddle method (Apparatus USP type II) by immersing the dosage form in 750 ml of 0.1 N HCl for 2 hours at a speed of 50 rpm and then adding 1000 ml of phosphate buffer to afford a pH of 6.8. Aliquots of the medium are withdrawn at various time points over a period of 24 hours. The aliquots are then analyzed using HPLC with UV detection to determine the concentration of dissolved active ingredient using standard methodology.

### EXAMPLE 14

TABLE 2
Comparative stability study between composition
of comparative example 1A and example 5
Percentage of total related substances (RS)

| | Comparative example 1A | Example 5 |
|---|---|---|
| Initial | 0.20 | 0.15 |
| 15 day 40° C./75% RH | 1.59 | 0.38 |

Stability of sustained release formulations of pregabalin with sucrose fatty acid ester as release controlling agent and with hydroxy propyl methyl cellulose as release controlling agent was comparatively evaluated. Stability tests were conducted under accelerated conditions for a period of 15 days at 40° C. and 75% RH .At the end of the specified time period formulations were analyzed for impurities resulting from degradation of pregabalin. The results of the stability analysis is listed in Table 2. The data shows that sustained release formulations of pregabalin embedded in a sucrose fatty acid ester matrix showed lesser degradation products as compared to HPMC matrices.

### EXAMPLE 15

A comparative in vivo bio availability study was conducted to evaluate the pharmacokinetic parameters of the compositions of present invention. Accordingly a bio study was conducted on 30 healthy volunteers under the fasting conditions using 2 compositions viz. compositions of comparative example 1A and example 5.

Area under the concentration-time curve (AUC) values were calculated from zero to infinite time using the linear trapezoidal rule

TABLE 3
AUC₀₋ₜ

| Parameter | Example 5 | Comparative Example 1A |
|---|---|---|
| N | 30 | 30 |
| Mean (μg/ml) | 56.900 | 35.590 |
| SD | 12.671 | 16.870 |
| Min | 33.804 | 7.144 |
| Median | 53.635 | 30.823 |
| Max | 83.160 | 85.649 |
| CV % | 22.300 | 47.400 |
| Geometric Mean | 55.557 | 31.765 |

TABLE 4
AUC_{0-inf}

| Parameter | Example 5 | Comparative Example 1A |
|---|---|---|
| N | 30 | 30 |
| Mean (μg/ml) | 58.451 | 37.032 |
| SD | 12.983 | 16.978 |
| Min | 34.990 | 9.210 |
| Median | 55.210 | 32.000 |
| Max | 86.350 | 86.980 |
| CV % | 22.200 | 45.800 |
| Geometric Mean | 57.078 | 33.406 |

From the above table it is observed that the percentage coefficient of variation for HPMC matrices is significantly higher as compared to sucrose fatty acid ester matrices. Thus it is inferred that pregabalin sustained release compositions using sucrose fatty acid esters as release retarding agents afforded less inter individual variability in AUC levels as compared with hydrophilic polymeric matrix systems such as HPMC compositions. Pregabalin sustained release formulations of comparative example 1A are released prematurely and in an exaggerated manner in the aqueous environment of the gastrointestinal tract. This invariably results in fluctuations of drug in the plasma which is reflected as inter individual variability in AUC of the treated subjects. In contrast pregabalin sustained release formulations comprising sucrose fatty acid ester as sustained release matrix ensured uniform release of drug throughout the gastrointestinal tract and stable plasma levels. Thus negligible amount of variability in seen in the AUC of subjects treated with pregabalin embedded in a sucrose fatty acid ester matrix.

### EXAMPLE 16

An open label, balanced, randomized, two treatment, two period, two sequence, crossover, comparative bioavailability study of once a day sustained release Pregabalin 300 mg tablet as described in example 5 herein with Lyrica® [(Pregabalin capsule 150 mg)×2 (1 capsule 12 hourly)] was conducted in 24 normal, healthy, adult, male, human subjects under fasting condition.

The venous blood samples were withdrawn at specific time intervals and plasma drug levels were monitored using a validated method and pharmacokinetic parameters Cmax, AUC_{0-t and} AUC_{0-inf} were evaluated.

TABLE 5

| | | | | 90% Confidence | |
|---|---|---|---|---|---|
| | | Lyrica ® 2 x | | Intervals | |
| | | 150 mg | | Lower | Upper |
| Sno | Parameters | capsules | Example 5 | limit | limit |
| 1 | Cmax (μg/ml) | 4.8431 | 4.8380 | 92.12 | 108.33 |
| 2 | AUCₜ (μg/ml) | 55.7073 | 49.4311 | 82.30 | 95.68 |
| 3 | AUC_{inf} (μg/ml) | 57.5873 | 50.8521 | 81.91 | 95.19 |

From the above data it is inferred that sustained release formulations of pregabalin according to the present invention are bioequivalent to conventional immediate release formulations of dose 150 mg pregabalin administered twice daily.

### EXAMPLE 17

An open label, balanced, randomized, two treatment, two period, two sequence, crossover, comparative bioavailability study of once a day sustained release Pregabalin 300 mg tablet as described in example 5 herein with Lyrica® [(Pregabalin capsule 100 mg)×3] was conducted in 30 normal, healthy, adult, male, human subjects under fasting condition.

The venous blood samples were withdrawn at specific time intervals and plasma drug levels were monitored using a validated method and pharmacokinetic parameters AUC_{0-t and} AUC_{0-inf} were evaluated.

TABLE 6

| | | | | 90% Confidence | |
|---|---|---|---|---|---|
| | | Lyrica ® 3 x | | Intervals | |
| | | 100 mg | | Lower | Upper |
| Sno | Parameters | capsules | Example 5 | limit | limit |
| 1 | AUC₀₋ₜ (μg/ml) | 57.154 | 55.557 | 86.69 | 86.30 |
| 2 | AUC_{0-inf} (μg/ml) | 59.457 | 57.078 | 109.00 | 106.78 |

From the above data it is inferred that sustained release formulations of pregabalin according to the present invention are comparable to conventional immediate release formulations of pregabalin of dose 100 mg administered thrice daily.

We claim:

1. A sustained release pharmaceutical composition adapted for once daily dosing, comprising pregabalin or a pharmaceutically acceptable salt, complex, solvate or hydrate thereof, release retarding agent(s) and optionally other pharmaceutically acceptable excipients thereof, wherein said pharmaceutical composition is bioequivalent to immediate release formulations of pregabalin administered twice daily.

2. A sustained release pharmaceutical composition according to claim 1, wherein the release retarding agent is selected from the group consisting of cellulose ethers, cellulose esters, acrylic acid copolymers, waxes, gums, glyceryl fatty acid esters and sucrose fatty acid esters.

3. A sustained release pharmaceutical composition according to claim 2, wherein the sucrose fatty acid ester is selected from the group comprising, sucrose stearate, sucrose distearate, sucrose palmitate, sucrose oleate, sucrose laurate, sucrose behenate, sucrose erucate and combinations thereof.

4. A sustained release pharmaceutical composition according to claim 1, wherein the composition comprises pregabalin in an amount from about 25% to about 75% by weight of the dosage form and release retarding agent in an amount from about 20% to about 80% by weight of the dosage form.

5. A sustained release pharmaceutical composition according to claim 1, comprising
a) 30% to 50% w/w of pregabalin or a pharmaceutically acceptable salt, complex, solvate or hydrate thereof
b) 30% to 60% w/w of sucrose fatty acid ester
c) Optionally 1%-50% w/w of pharmaceutically acceptable excipients

6. A sustained release pharmaceutical composition according to claim 1, wherein the composition exhibits an in vitro release of atleast about 25% of pregabalin after 6 hours and atleast about 50% of pregabalin after 12 hours and in an aqueous medium.

7. A sustained release pharmaceutical compositions according to claim 1, in which the compositions exhibits an in vitro release of at least about 75% of pregabalin after 24 hours in an aqueous medium.

8. A process for the preparation of a pharmaceutical composition according to claim 2, wherein the process comprises mixing pregabalin; sucrose fatty acid ester and optionally other pharmaceutical excipients, granulating the resultant blend compressing the granules into a tablet or filling into a hard gelatin capsule.

9. A process for the preparation of a pharmaceutical composition according to claim 8 comprising the following steps:
Step 1. Mixing pregabalin; sucrose fatty acid ester and optionally other pharmaceutical excipients,
Step 2. Making slugs of resultant mixture as obtained in step 1
Step 3. Breaking down the slugs obtained in step 3 to obtain the granules of desired size
Step 4. Optionally mixing the granules of step 3 with other excipients to obtain final blend.
Step 5. Compressing the resultant blend of step 4 to obtain tablets or filling such final blend into hard gelatin capsules.

10. A sustained release composition according to claim 1, wherein pharmaceutical composition administered once daily exhibits an AUC of pregabalin ranging from 10 μg/ml to 100 μg/ml.

11. A sustained release composition according to claim 10, wherein pharmaceutical composition administered once daily exhibits an AUC of pregabalin ranging from 30 μg/ml to 80 μg/ml.

12. A sustained release composition according to claim 10, wherein pharmaceutical composition administered once daily exhibits an AUC of pregabalin ranging from 40 μg/ml to 60 μg/ml.

13. A sustained release composition according to claim 1, wherein pharmaceutical composition administered once daily provides peak plasma concentrations of pregabalin ranging from 1 μg/ml to 10 μg/ml.

14. A sustained release composition according to claim 13, wherein pharmaceutical composition administered once daily provides peak plasma concentrations of pregabalin ranging from 2.5 μg/ml to 8 μg/ml.

15. A sustained release composition according to claim 13, wherein pharmaceutical composition administered once daily provides peak plasma concentrations of pregabalin ranging from 3.5 μg/ml to 6 μg/ml.

16. A sustained release pharmaceutical composition adapted for once daily dosing, comprising pregabalin or a pharmaceutically acceptable salt, complex, solvate or hydrate thereof, release retarding agent(s) and optionally other pharmaceutically acceptable excipients thereof, wherein said pharmaceutical composition is bioequivalent when compared in terms of total exposure (AUC) to conventional immediate release composition of pregabalin administered thrice daily.

17. A sustained release composition as herein above described in the specification with reference to the accompanying drawing.
